(19) United States
(12) Patent Application Publication
(10) Pub. No.: US 2021/0145775 A1
(43) Pub. Date: May 20, 2021

(54) Treatment of intestinal barrier dysfunction and associated diseases

(71) Applicant: CymaBay Therapeutics, Inc., Newark, CA (US)

(72) Inventor: Edward E. Cable, San Leandro, CA (US)

(73) Assignee: CymaBay Therapeutics, Inc., Newark, CA (US)

(21) Appl. No.: 17/096,385

(22) Filed: Nov. 12, 2020

Related U.S. Application Data
(60) Provisional application No. 62/935,459, filed on Nov. 14, 2019.

(51) Int. Cl. A61K 31/192; A61K 9/00; A61P 1/00

(52) U.S. Cl. CPC A61K 31/192; A61P 1/00; A61K 9/0053

(57) ABSTRACT

Treatment of intestinal barrier dysfunction and associated diseases other than alcoholic liver disease or NAFLD/NASH with seladelpar or a salt thereof.

### CROSS-REFERENCE TO RELATED APPLICATION

This application claims the benefit under 35 USC 119(e) of Application No. 62/935459, filed 14 Nov. 2019, the entire content of which is incorporated into this application by reference.

### FIELD OF THE INVENTION

This invention relates to the treatment of intestinal barrier dysfunction and associated diseases other than alcoholic liver disease or NAFLD/NASH.

### DESCRIPTION OF THE RELATED ART

The intestinal barrier

According to Assimakopoulos et al., “The Role of the Gut Barrier Function in Health and Disease”, Gastroenterol. Res., 11(4), 261-263 (2018) [internal citations omitted]: “The intestinal tract contains the body's largest interface between a person and his or her external environment. The complexity of its function is obvious when thinking that at the same time the intestine must serve two opposite functions; the selective permeability of needed nutrients from the intestinal lumen into the circulation and into the internal milieu in general and, on the other hand, the prevention of the penetration of harmful entities including microorganisms, luminal antigens, and luminal proinflammatory factors. The latter function is known as barrier function. The gut barrier function is comprised by three major lines of defence: 1) The biological barrier, which is made up of normal intestinal flora (gut microbiota) responsible for colonization resistance; 2) The immune barrier, which is composed of gut associated lymphoid tissue (GALT), effector and regulatory T cells, IgA producing B (plasma) cells, group 3 innate lymphoid cells, and, resident macrophages and dendritic cells in the lamina propria; and 3) The mechanical barrier, consisting of the closed-lining intestinal epithelial cells and by the capillary endothelial cells. . . . The term ‘bacterial translocation’ (BT), was first described by Berg and Garlington in 1979, as the phenomenon of passage of viable bacteria from the gastrointestinal tract through the epithelial mucosa into the lamina propria and then to the mesenteric lymph nodes and possibly other normally sterile organs. This initial definition was later widened to include the translocation of non-viable bacteria or their products, namely pathogen-associated molecular patterns (PAMPs), with main representative the intestinal endotoxin. BT occurs in healthy individuals in a low rate of 5-10%, serving two main physiological roles; the antigenic exposure of the gut immune system to be prepared for an effective immune response in case of extensive pathogen invasion, and the development of immune tolerance to several microbial antigens of commensal microflora.”

The intestinal barrier is also referred to as the intestinal epithelial barrier, the intestinal mucosal barrier, the gut vascular barrier, and the gut barrier, among other terms; and “intestinal barrier” includes any of these essentially synonymous terms. “Intestinal barrier dysfunction” refers to a decrease in function of the intestinal barrier, including essentially synonymous terms such as “intestinal barrier loss”, “gut vascular barrier disruption” and the like.

Diseases associated with intestinal barrier dysfunction

Assimakopoulos et al. also note: “The intestinal barrier is compromised in several disease states leading to an increased level of BT associated with infectious complications and promotion of a systemic inflammatory response that aggravates the pathophysiological consequences of the underlying disease. There are three main pathophysiological groups of intestinal barrier failure associated with pathologic conditions:

1) The intestinal barrier failure observed in surgical patients subjected to major operations for diverse reasons (major liver resections, bowel resections for malignancy, bowel transplantation, aortic aneurysm repair). In this group of patients, increased BT is associated with increased postoperative infectious complications. The connecting mechanism is translocation of gut-derived pathogens through a dysfunctional mucosal barrier to the mesenteric lymph nodes, the portal vein and the systemic circulation, eventually leading to postoperative infections. Also, this is the mechanism by which the necrotic pancreas becomes infected in patients with severe necrotic pancreatitis.

2) The second group includes critically ill patients, severely injured or septic, hospitalized in intensive care units. Increased gut permeability is associated with the development of systemic inflammatory response and multiple organ dysfunction syndrome (MODS) in these patients. However, the connecting pathophysiological link of gut barrier failure and MODS does not seem to be the classical process of BT. Current pathogenetic aspects support the ‘gut-lymph’ theory of sepsis and MODS. According to this theory, microbes and/or their products, through a dysfunctional gut barrier, first gain access to the intestinal submucosa activating the intestinal immunological system of defence. An intestinal proinflammatory response further aggravates intestinal injury and danger associated molecular patterns (DAMPs) are released in the mesenteric lymphatics, carried to the lung and the systemic circulation, stimulating Toll like receptors-4 and perhaps other pattern recognition receptors (PRR) in a fashion similar to bacteria, thus eventually promoting injurious effects in diverse organs. Therefore, the gut becomes a pivotal proinflammatory organ promoting deleterious effects in even distant organs, through release of DAMPs, without the need of systemic bacterial translocation.

3) The third group of intestinal barrier dysfunction involves stable patients with chronic pathologic conditions that present a low-grade translocation of enteric microbes and immunostimulatory bioproducts from the gut lumen first in the lamina propria and thereafter in the systemic circulation, promoting a chronic immune activation associated with disease progression and/or development of complications and comorbidities from other organs. This intestinal barrier dysfunction group encompasses patients with HIV infection, liver cirrhosis, chronic viral hepatitis B or C, non-alcoholic steatohepatitis or non-alcoholic fatty liver disease, patients with inflammatory bowel diseases, celiac disease, irritable bowel syndrome, obesity and diverse autoimmune conditions. For example, in HIV infection, intestinal barrier dysfunction, BT and chronic immune activation have been associated with cardiovascular, neurocognitive and lymphoproliferative comorbidities, despite effective viral suppression with modern antiretroviral treatment; and in liver cirrhosis intestinal barrier dysfunction has been associated with all of its complications, namely spontaneous bacterial peritonitis, hepatic encephalopathy, hepatorenal syndrome, hepatopulmonary syndrome, variceal bleeding, progression of liver injury and hepatocellular carcinoma.”

A discussion of intestinal barrier function and dysfunction is given by Groschwitz et al., “Intestinal barrier function: Molecular regulation and disease pathogenesis”, J. Allergy Clin. Immunol., 124(1), 3-20 (2009). Catalioto et al., “Intestinal Epithelial Barrier Dysfunction in Disease and Possible Therapeutical Interventions”, Curr. Med. Chem., 18(3), 398-426 (2011), list the following diseases as being associated with increased intestinal permeability: the intestinal disorders of inflammatory bowel disease (IBD), Crohn's disease, ulcerative colitis, irritable bowel syndrome, collagenous colitis, intestinal ischemia, chemotherapy-induced mucositis, NSAID-induced enteropathy, intestinal infections, sepsis, celiac disease and food allergy (where reduced intestinal barrier function correlates with the severity of symptoms experienced by food allergy sufferers); and the non-intestinal disorders of alcoholic liver disease, nonalcoholic liver disease, total parenteral nutrition, Type 1 diabetes, acute pancreatitis, chronic heart failure, emotional stress, and multiple organ dysfunction syndrome. Fukui, “Increased Intestinal Permeability and Decreased Barrier Function: Does It Really Influence the Risk of Inflammation?”, Inflamm. Intest. Dis., 1, 135-145 (2016), include obstructive jaundice, acute pancreatitis, chronic kidney disease, chronic heart failure, and depression as conditions that may be influenced by intestinal barrier dysfunction. Nalle et al., “Intestinal barrier loss as a critical pathogenic link between inflammatory bowel disease and graft-versus-host disease”, Mucosal Immunol., 8(4), 720-730 (2015), also link intestinal barrier loss with rheumatoid arthritis and multiple sclerosis; and note that evidence suggests a connection between intestinal barrier loss and graft-versus-host disease (GVHD), pointing to similarities between IBD and GVHD.

Thus intestinal barrier dysfunction (increased intestinal permeability) is a significant factor in many diseases.

Treatments for intestinal barrier dysfunction

Catalioto et al. mention as “potential therapies to restore intestinal barrier function” biological therapies based on growth factor receptor activation (mentioning, for example, the GLP-2 peptide tedeglutide), probiotics, the zonulin antagonist larazotide [currently under Phase 3 study in celiac disease], and various NF-κB pathway inhibitors.

However, treatments for the various diseases considered as associated with intestinal barrier dysfunction tend to be relatively specific for the disease rather than simply being general for intestinal barrier dysfunction as such. Thus, although such interventions as dietary restriction are common in a number of intestinal disorders, such interventions are not common in non-intestinal disorders. For example, treatment for IBS is primarily diet, however, several motility-controlling drugs such as alosetron, rifaximin, and lubiprostone are approved to treat IBS; while treatment for IBD includes anti-inflammatory drugs like aminosalicylates and corticosteroids, immune system suppressors (e.g., azathioprine, cyclosporine, and methotrexate), and antibiotics (metronidazole or ciprofloxacin). Similarly, treatments for celiac disease and food allergies primarily involve avoidance of the allergen, though the use of steroids to control inflammation and immunomodulators such as azathioprine and tofactitinib are also used in more severe cases. For ulcerative colitis and Crohn's disease, 5-aminosalicylic acid in various forms (sulfasalazine, mesalamine, balsalazide, and olsalazine) is a common first treatment, with corticosteroids, immunomodulators, and biologics such as anti-tumor necrosis factor (TNF) inhibitors such as infliximab, adalimumab, and golimumab; and vedoluzimab (which is gut-specific) may be used in more advanced cases. GVHD is commonly treated with immunosuppressive drugs.

There exists a need for effective and tolerable therapy for intestinal barrier dysfunction and associated diseases.

Seladelpar

Seladelpar (International Nonproprietary Name—INN) has the chemical name [4-({(2R)-2-ethoxy-3-[4-(trifluoromethyl)phenoxy]propyl }sulfanyl)-2-methylphenoxylacetic acid [IUPAC name from WHO Recommended INN: List 77], and the code number MBX-8025. Seladelpar and its synthesis, formulation, and use is disclosed in, for example, U.S. Pat. No. 7,301,050 (compound 15 in Table 1, Example M, claim 49), U.S. Pat. No. 7,635,718 (compound 15 in Table 1, Example M), and U.S. Pat. No. 8,106,095 (compound 15 in Table 1, Example M, claim 14). Lysine (L-lysine) salts of seladelpar and related compounds are disclosed in U.S. Pat. No. 7,709,682 (seladelpar L-lysine salt throughout the Examples, crystalline forms claimed).

Seladelpar is an orally active, potent (2 nM) agonist of peroxisome proliferator-activated receptor-δ (PPARδ). It is specific (>600-fold and >2500-fold compared with PPARα and peroxisome proliferator-activated receptor-γ receptors). PPARδ activation stimulates fatty acid oxidation and utilization, improves plasma lipid and lipoprotein metabolism, glucose utilization, and mitochondrial respiration, and preserves stem cell homeostasis. According to U.S. Pat. No. 7,301,050, PPARδ agonists, such as seladelpar, are suggested to treat PPARδ-mediated conditions, including “diabetes, cardiovascular diseases, Metabolic X syndrome, hypercholesterolemia, hypo-high density lipoprotein (HDL)-cholesterolemia, hyper-low density lipoprotein (LDL)-cholesterolemia, dyslipidemia, atherosclerosis, and obesity”, with dyslipidemia said to include hypertriglyceridemia and mixed hyperlipidemia.

U.S. Pat. No. 9,486,428 and PCT International Publication No. WO 2015/143178 disclose the treatment of intrahepatic cholestatic diseases, such as primary biliary cholangitis, primary sclerosing cholangitis, progressive familial intrahepatic cholestasis, and Alagille syndrome, with seladelpar and its salts; U.S. Pat. No. 9,381,181 and PCT International Publication No. WO 2015/157697 disclose the treatment of non-alcoholic fatty liver disease and non-alcoholic steatohepatitis with seladelpar and its salts; US Application Publication No. 2015-0374649 and PCT International Publication No. WO 2015/200580 disclose the treatment of severe hypertriglyceridemia with seladelpar and its salts; and US Application Publication No. 2015-0139987 and PCT International Publication No. WO 2015/077154 disclose the treatment of homozygous familial hypercholesterolemia with seladelpar and its salts.

Seladelpar has also been studied in primary biliary cholangitis (PBC), with results for 50 and 200 mg/day reported in Jones et al., “Seladelpar (MBX-8025), a selective PPAR-δ agonist, in patients with primary biliary cholangitis with an inadequate response to ursodeoxycholic acid: a double-blind, randomised, placebo-controlled, phase 2, proof-of-concept study”, Lancet Gastroenterol. Hepatol., 2(10), 716-726 (2017), and for 2, 5, and 10 mg/day at The International Liver Congress™ hosted by the European Association for the Study of Liver Diseases (EASL) in Paris, France (Apr. 11-15, 2018): in poster LBP-2 (Hirschfield et al., “Treatment Efficacy and Safety of Seladelpar, a Selective Peroxisome Proliferator-Activated Receptor Delta agonist, in Primary Biliary Cholangitis Patients: 12- and 26-Week Analyses of an Ongoing, International, Randomized, Dose Ranging Phase 2 Study”), and in poster THU-239 (Boudes et al., “Seladelpar's Mechanism of Action as a Potential Treatment for Primary Biliary Cholangitis and Non-Alcoholic Steatohepatitis”), both available at https://ir.cymabay.com/presentations.

Haczeyni et al., “The Selective Peroxisome Proliferator-Activated Receptor-Delta Agonist Seladelpar Reverses Nonalcoholic Steatohepatitis Pathology by Abrogating Lipotoxicity in Diabetic Obese Mice”, Hepatol. Comm., 1(7), 663-674 (2017), have reported that seladelpar improves NASH pathology (reducing hepatic steatosis and inflammation, and improving fibrosis) in atherogenic diet-fed obese diabetic (Alms1 mutant (foz/foz)) mice, a well-known animal model for human NAFLD/NASH. Choi et al., “Seladelpar Improves Hepatic Steatohepatitis and Fibrosis in a Diet-Induced and Biopsy-Confirmed Mouse Model of NASH”, Abstract 1311 for the Liver Meeting® 2018 of the American Association for the Study of Liver Diseases (AASLD), have reported similar results in atherogenic diet-fed normal mice (DIO-NASH). CymaBay Therapeutics has completed a Phase 2b study of seladelpar in patients with NASH using doses of 10, 20, and 50 mg/day, NCT03551522: see CymaBay press release “CymaBay Therapeutics Announces the Initiation of a Phase 2b Study of Seladelpar in Patients with Non-Alcoholic Steatohepatitis”, https://ir.cymabay.com/pres s-releases/detail/431/cymabay-therapeutics-announces-the-initiation-of-a-phase-2b-study-of-seladelpar-in-patients-with-non-alcoholic-steatohepatitis.

The entire disclosures of the documents referred to in this application are incorporated into this application by reference.

### SUMMARY OF THE INVENTION

This invention is the treatment of intestinal barrier dysfunction and associated diseases other than alcoholic liver disease or NAFLD/NASH by administration of seladelpar or a salt thereof.

In view of the demonstrated efficacy of seladelpar in stabilizing intestinal barrier function in a mouse model of alcoholic liver disease, seladelpar is expected to have activity in treating intestinal barrier dysfunction and therefore also be useful for treating diseases, other than alcoholic liver disease or NAFLD/NASH, that are also associated with intestinal barrier dysfunction.

Preferred aspects of this invention are characterized by the specification and by the features of claims 1 to 20 of this application as filed.

### DETAILED DESCRIPTION OF THE INVENTION

Definitions

The intestinal barrier, diseases associated with intestinal barrier dysfunction, and treatments for intestinal barrier dysfunction are described in the subsections entitled “The intestinal barrier”, “Diseases associated with intestinal barrier dysfunction” and “Treatments for intestinal barrier dysfunction” of the DESCRIPTION OF THE RELATED ART.

“Treating” or “treatment” of intestinal barrier dysfunction and/or a disease associated with intestinal barrier dysfunction other than alcoholic liver disease or NAFLD/NASH includes one or more of:
(1) preventing or reducing the risk of the manifestations of intestinal barrier dysfunction or one of its associated diseases such as irritable bowel syndrome, or reducing the risk of consequences of those manifestations, such as reducing the risk of abdominal pain; i.e., causing the manifestations of intestinal barrier dysfunction or an associated disease such as irritable bowel syndrome, or the consequences such as abdominal pain, not to develop in a subject who may be suffering from intestinal barrier dysfunction or an associated disease but who does not yet experience or display the manifestations of that condition (i.e. prophylaxis);
(2) inhibiting the manifestations of intestinal barrier dysfunction or one of its associated diseases, i.e., arresting or reducing the development of the manifestations; and
(3) relieving the manifestations of intestinal barrier dysfunction or one of its associated diseases, i.e., reducing the number, frequency, duration or severity of the manifestations.

A “therapeutically effective amount” of seladelpar or a seladelpar salt means that amount which, when administered to a human for treating intestinal barrier dysfunction or one of its associated diseases, is sufficient to effect treatment for the disease. The therapeutically effective amount for a particular subject varies depending upon the age, health and physical condition of the subject to be treated, the disease and its extent, the assessment of the medical situation, and other relevant factors. It is expected that the therapeutically effective amount will fall in a relatively broad range that can be determined through routine trial.

Seladelpar is described in the subsection entitled “Seladelpar” of the DESCRIPTION OF THE RELATED ART.

Salts (for example, pharmaceutically acceptable salts) of seladelpar are included in this invention and are useful in the methods described in this application. These salts are preferably formed with pharmaceutically acceptable acids. See, for example, “Handbook of Pharmaceutically Acceptable Salts”, Stahl and Wermuth, eds., Verlag Helvetica Chimica Acta, Zürich, Switzerland, for an extensive discussion of pharmaceutical salts, their selection, preparation, and use. Unless the context requires otherwise, reference to seladelpar is a reference both to the compound and to its salts.

Because seladelpar contains a carboxyl group, it may form salts when the acidic proton present reacts with inorganic or organic bases. Typically, seladelpar is treated with an excess of an alkaline reagent, such as hydroxide, carbonate or alkoxide, containing an appropriate cation. Cations such as Na⁺, K⁺, Ca²⁺, Mg²⁺, and NH₄⁺ are examples of cations present in pharmaceutically acceptable salts. Suitable inorganic bases, therefore, include calcium hydroxide, potassium hydroxide, sodium carbonate and sodium hydroxide. Salts may also be prepared using organic bases, such as salts of primary, secondary and tertiary amines, substituted amines including naturally-occurring substituted amines, and cyclic amines, including isopropylamine, trimethylamine, diethylamine, triethylamine, tripropylamine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, N-ethylpiperidine, and the like. Useful salts are expected to include the L-lysine salts; and, as noted in the “Seladelpar” subsection, seladelpar is currently formulated as its L-lysine dihydrate salt.

“Comprising” or “containing” and their grammatical variants are words of inclusion and not of limitation and mean to specify the presence of stated components, groups, steps, and the like but not to exclude the presence or addition of other components, groups, steps, and the like. Thus “comprising” does not mean “consisting of”, “consisting substantially of”, or “consisting only of”; and, for example, a formulation “comprising” a compound must contain that compound but also may contain other active ingredients and/or excipients.

Formulation and administration

Seladelpar may be administered by any route suitable to the subject being treated and the nature of the subject's condition. Routes of administration include administration by injection, including intravenous, intraperitoneal, intramuscular, and subcutaneous injection, by transmucosal or transdermal delivery, through topical applications, nasal spray, suppository and the like or may be administered orally. Formulations may optionally be liposomal formulations, emulsions, formulations designed to administer the drug across mucosal membranes or transdermal formulations. Suitable formulations for each of these methods of administration may be found, for example, in “Remington: The Science and Practice of Pharmacy”, 20th ed., Gennaro, ed., Lippincott Williams & Wilkins, Philadelphia, Pa., U.S.A. Because seladelpar is orally available, typical formulations will be oral, and typical dosage forms will be tablets or capsules for oral administration. As mentioned in the “Seladelpar” subsection, seladelpar has been formulated in capsules for clinical trials. Intravenous formulations may be particularly applicable for administration to acutely ill subjects, such as subjects suffering from acute alcoholic hepatitis or alcoholic fibrosis or cirrhosis, such as those subjects who may be hospitalized for treatment.

Depending on the intended mode of administration, the pharmaceutical compositions may be in the form of solid, semi-solid or liquid dosage forms, preferably in unit dosage form suitable for single administration of a precise dosage. In addition to an effective amount of seladelpar, the compositions may contain suitable pharmaceutically-acceptable excipients, including adjuvants which facilitate processing of the active compounds into preparations which can be used pharmaceutically. “Pharmaceutically acceptable excipient” refers to an excipient or mixture of excipients which does not interfere with the effectiveness of the biological activity of the active compound(s) and which is not toxic or otherwise undesirable to the subject to which it is administered.

For solid compositions, conventional excipients include, for example, pharmaceutical grades of mannitol, lactose, starch, magnesium stearate, sodium saccharin, talc, cellulose, glucose, sucrose, magnesium carbonate, and the like. Liquid pharmacologically administrable compositions can, for example, be prepared by dissolving, dispersing, etc., an active compound as described herein and optional pharmaceutical adjuvants in water or an aqueous excipient, such as, for example, water, saline, aqueous dextrose, and the like, to form a solution or suspension. If desired, the pharmaceutical composition to be administered may also contain minor amounts of nontoxic auxiliary excipients such as wetting or emulsifying agents, pH buffering agents and the like, for example, sodium acetate, sorbitan monolaurate, triethanolamine sodium acetate, triethanolamine oleate, etc.

For oral administration, the composition will generally take the form of a tablet or capsule; or, especially for pediatric use, it may be an aqueous or nonaqueous solution, suspension or syrup. Tablets and capsules are preferred oral administration forms. Tablets and capsules for oral use will generally include one or more commonly used excipients such as lactose and corn starch. Lubricating agents, such as magnesium stearate, are also typically added. When liquid suspensions are used, the active agent may be combined with emulsifying and suspending excipients. If desired, flavoring, coloring and/or sweetening agents may be added as well. Other optional excipients for incorporation into an oral formulation include preservatives, suspending agents, thickening agents, and the like.

Typically, a pharmaceutical composition of seladelpar, or a kit comprising compositions of seladelpar, is packaged in a container with a label, or instructions, or both, indicating use of the pharmaceutical composition or kit in the treatment of intestinal barrier dysfunction or an associated disease.

A person of ordinary skill in the art of pharmaceutical formulation will be able to prepare suitable pharmaceutical compositions of the seladelpar by choosing suitable dosage forms, excipients, packaging, and the like, to achieve therapeutically effective formulations without undue experimentation and in reliance upon personal knowledge and the disclosure of this application.

A suitable amount of seladelpar or a salt thereof for oral dosing for an adult subject with intestinal barrier dysfunction or an associated disease, depending on the extent and severity of the disease, and factors such as hepatic and renal function, is expected to be between 1 and 200 mg/day, preferably between 5 and 100 mg/day, such as 5, 10, 20, 50, or 100 mg/day, when the amount is calculated as seladelpar. “When the amount is calculated as seladelpar” means that if a seladelpar salt is being used, the amount of that salt will be the amount that is equivalent to the stated amount of seladelpar; for example, if seladelpar L-lysine dihydrate salt is being used, the amount will be multiplied by the formula weight of seladelpar L-lysine dihydrate salt divided by the formula weight of seladelpar, or about 1.41; so that an amount of 100 mg/day when the amount is calculated as seladelpar will require an amount of about 141 mg/day of seladelpar L-lysine dihydrate salt. That is, a suitable amount of seladelpar for oral dosing is expected to be similar to the amounts employed in clinical trials for PBC, PSC, NASH, and other conditions. Suitable reductions in dose toward or below the lower end of the outer range above will be made for subjects who are children, depending on such additional factors as age and body mass; and in subjects with significant hepatic impairment, such as subjects in Child-Pugh classes B and C, depending on the degree of impairment. These amounts represent an average daily dose, and not necessarily an amount given at a single dose. Dosing may be as frequent as more than once/day (where the amount, or daily dose, will be divided between the number of administrations per day), but will more typically be once/day (where the amount is given in a single administration). Optionally, particularly in cases of significant hepatic impairment, the dosing may be less frequent than once/day, such as between once/week and every other day, for example once/week, twice/week (especially with the doses at least three days apart), three times/week (especially with the doses at least two days apart), or every other day. Similar amounts and dosing schedules are expected to be applicable for dosing by injection, such as for intravenous administration

A person of ordinary skill in the art of the treatment of intestinal barrier dysfunction and associated diseases, who will typically be a person of ordinary skill in the art of the treatment of intestinal and hepatobiliary diseases, will be able to ascertain a therapeutically effective amount of seladelpar for a particular extent of disease and patient to achieve a therapeutically effective amount for the treatment of intestinal barrier dysfunction and associated diseases without undue experimentation and in reliance upon personal knowledge, the skill of the art, and the disclosure of this application.

### EXAMPLES

### Example 1

### Preclinical, Lieber-DeCarli Ethanol Diet

Age-matched wild-type female C57BL/6 mice (Charles River, Wilmington, Mass.), 8-9 weeks old at the initiation of the study, were used. The mice were divided into five study groups with initial numbers as follows:
(1) control diet (n=10);
(2) control diet with seladelpar prevention (n=10);
(3) ethanol diet (n=24);
(4) ethanol diet with seladelpar prevention (n=24); and
(5) ethanol diet with seladelpar intervention (n=24).

The Lieber-DeCarli ethanol diet was originally developed by Charles Lieber and Leonore DeCarli in 1963. This diet allows for the prolonged exposure of ethanol in a rodent model and allows for modification to calories provided by ethanol. The Lieber-DeCarli ethanol diet used consisted of PMI® Micro Stabilized Alcohol Rodent Liquid Diet LD101A and PMI® Maltodextrin LD104, both from TestDiet (St. Louis, Mo.) and 200 proof ethanol from Gold Shield (Hayward, Calif.) in a specific combination following TestDiet's preparation and feeding directions [https://www.testdiet.com/cs/-groups/lolweb/@testdiet/documents/web_content/mdrf/mdi2/˜edisp/ducm04_026403.pdf]. The caloric intake from ethanol was 0% on day 1, 10% on days 2 and 3, 20% on days 4 and 5, 30% from day 6 until the end of 6 weeks, and 36% for the last 2 weeks, for a total in-life time of eight weeks. The ethanol-fed mice also received one bolus dose of 33% v/v ethanol at 5 g/Kg on the last in-life day. The control diet contained an isocaloric substitution of isomaltose for the ethanol. Where appropriate, seladelpar (as a solution made from the L-lysine dihydrate salt) was added at 0.015 mg seladelpar/mL to the prepared liquid diet: assuming a consumption of about 0.68 mL/day, this resulted in an oral dose of seladelpar of 10 mg/Kg/day. In the prevention portions of the study, including with the control diet, seladelpar was added throughout the entire study period; in the intervention portion of the study, seladelpar was added only for the fifth through eighth weeks. Fresh feces were collected at 7.5 weeks. All mice were sacrificed after eight weeks; the ethanol-fed mice eight hours after the ethanol bolus. At sacrifice, body and liver weight were recorded; and blood/plasma, liver, gallbladder, small and large intestine (wall and contents) and cecum were harvested. Analyses included plasma ALT—to measure inflammation; hepatic TGs; plasma lipopolysaccharides (LPS)—to measure the extent of bacterial leakage from the intestine into the blood; fecal albumin (FA)—to measure the extent of albumin leakage from the blood into the intestine (feces); and total bile acids, measured by liquid chromatography-mass spectrometry (LC-MS) generally according to the method described in Hartmann et al., “Modulation of the Intestinal Bile Acid/Farnesoid X Receptor/Fibroblast Growth Factor 15 Axis Improves Alcoholic Liver Disease in Mice”, Hepatology, 67(6), 2150-2166 (2018). Plasma ethanol was also measured in the ethanol group, and did not vary significantly between groups 3, 4, and 5.

The study results are given in the table below: standard errors of the means are in parentheses, n denotes the number of mice measured, and ANOVA denotes the significance value of an ANOVA analysis between groups 3, 4, and 5:

| Study group | 1 | 2 | 3 | 4 | 5 | ANOVA |
|---|---|---|---|---|---|---|
| ALT (U/L) | 17.9 (5.7) | 20.8 (3.5) | 83.3 (16.7) | 37.8 (4.1) | 30.9 (4.1) | <0.001 |
| | n = 10 | n = 10 | n = 19 | n = 24 | n = 22 | |
| Hepatic TGs | 21.7 (1.5) | 18.9 (1.8) | 29.6 (2.0) | 21.8 (1.3) | 20.5 (1.3) | <0.001 |
| (mg/g) | n = 10 | n = 10 | n = 19 | n = 24 | n = 22 | |
| Plasma LPS | 645 (93) | 787 (108) | 1064 (204) | 723 (66) | 634 (59) | 0.034 |
| (ng/mL) | n = 10 | n = 10 | n = 18 | n = 23 | n = 22 | |
| FA (ng/mg) | 104 (12) | 95 (12) | 135 (15) | 98 (8) | 112 (9) | 0.060 |
| | n = 10 | n = 10 | n = 19 | n = 21 | n = 22 | |
| Total bile | 17.8 (1.6) | 14.2 (0.6) | 23.7 (1.6) | 16.5 (1.1) | 16.0 (1.1) | <0.001 |
| acids | n = 10 | n = 10 | n = 19 | n = 21 | n = 22 | |
| (μmol/100 g | | | | | | |
| body weight) | | | | | | |

Seladelpar prevention did not significantly affect any of the above parameters in the control diet groups; but ANOVA analysis showed significant reductions (improvements) in serum ALT, hepatic TGs, serum LPS, and total bile acids between the untreated group 3 and the seladelpar-treated groups 4 and 5 (i.e., both the prevention and intervention groups) in the alcohol diet groups. Although ANOVA analysis of fecal albumin was not significant at the p<0.05 level, there was a significant reduction (improvement) in fecal albumin between the untreated group 3 and the seladelpar-treated groups 4 and 5.

Formalin-fixed liver sections were stained for histological examination with hematoxylin and eosin (H+E). Examination of representative sections showed no significant change on seladelpar treatment for mice on the control diet (comparing groups 1 and 2); however, significant reduction of steatosis and restoration of a more normal architecture was seen on seladelpar treatment (both prevention and intervention) in the alcohol diet groups (comparing group 3 with groups 4 and 5).

This study demonstrated the benefit of seladelpar, in either prevention or intervention mode, in a model considered predictive for treatment of intestinal barrier dysfunction and associated diseases.

### Example 2

### Preclinical, Adoptive T-Cell Transfer Colitis

Adoptive transfer of CD4+ naïve T cells of normal mice into mutant Rag2 knockout mice on the same strain background has been shown to cause an acute inflammation in the colon, with gross and histopathologic changes resembling those occurring in Crohn's disease and ulcerative colitis in humans (see, for example, Ostanin et al., “T-cell transfer model of chronic colitis: concepts, considerations, and tricks of the trade”, Am. J. Physiol. Gastrointest. Liver Physiol., 296(2), G135-G146 (2009)); and the use of this predictive model is well documented. The cell donor animals are male C57BL/6 mice and the recipient animals are male RAG2^{-/-} mice on C57BL/6 background, aged 6-8 weeks at the start of the study. Recipient animals (20 +12 per test compound) are assigned: 5 to form a control group receiving no cell transfer or treatment, and the remainder to receive cell transfer. At day 0, the donor animals are sacrificed and spleens excised, dissociated to a single cell suspension, and the red blood cells lysed. The cell suspension is enriched for CD4+ T cells using a Miltenyi CD4+ T cell isolation kit, then further enriched for naïve CD4+ T cells using a Miltenyi naïve CD4+ T cell isolation kit. Non-control recipient animals receive an intraperitoneal injection of 0.5×10⁶ naïve T cells to induce colitis. On day 13, blood is collected by retroorbital eye bleed from the cell transfer recipient animals, and the animals are randomized into groups (15 to receive vehicle treatment, and 12 per test compound to receive compound treatment) by frequency of CD4+ events in the pelleted blood. Treatment begins on day 14 and continues to day 42. In the seladelpar treatment group, the animals receive 10 mg/Kg/day of seladelpar (as a solution made from the L-lysine dihydrate salt) in their drinking water. The animals are weighed daily and assessed visually for presence and severity of diarrhea and bloody stools, with video endoscopy on days 14, 28, and 42 to assess the severity of the colitis and score stool consistency. All animals are sacrificed at day 42 following endoscopy and samples collected: blood is collected and processed for plasma, the colon is excised, measured, rinsed, weighed, and trimmed to the most distal 5 cm, of this distal and proximal 2 cm portions are placed in formalin for histological evaluation, while the central 1 cm is snap-frozen in liquid nitrogen, and stored for analysis; and the liver is excised, weighed, snap-frozen in liquid nitrogen, and stored for analysis. While transfer of CD4+ naïve T cells induces colitis, to be seen by change in body weight and observed on endoscopic examination during the study, and by histologic examination post-mortem, seladelpar treatment ameliorates the colitis.

### Example 3

### Clinical (Oral)

Subjects with diagnosed irritable bowel syndrome (IBS) are treated with seladelpar or a salt thereof at 2 mg/day, 5 mg/day, 10 mg/day, 20 mg/day, 50 mg/day, or 100 mg/day, when the amount is calculated as seladelpar, for 12 weeks. The subjects keep a health diary in which their symptoms of irritable bowel syndrome are recorded. The subjects record a general improvement in their IBS symptoms (e.g. fewer and less severe instances of abdominal pain and cramping, diarrhea, or constipation), indicating a reduction in the manifestations of IBS.

The seladelpar-treated subjects show a dose-related improvement in their disease, as manifested by earlier and better management of the IBS, for example, reduction in gastrointestinal symptoms such as diarrhea.

While this invention has been described in conjunction with specific embodiments and examples, it will be apparent to a person of ordinary skill in the art, having regard to that skill and this disclosure, that equivalents of the specifically disclosed materials and methods will also be applicable to this invention; and such equivalents are intended to be included within the following claims.

1. A method of treating intestinal barrier dysfunction or an associated disease other than alcoholic liver disease or NAFLD/NASH by administering a therapeutically effective amount of seladelpar or a salt thereof.

2. The method of claim 1 where the seladelpar or a salt thereof is a seladelpar L-lysine salt.

3. The method of claim 2 where the seladelpar L-lysine salt is seladelpar L-lysine dihydrate salt.

4. The method of claim 1 where the seladelpar or a salt thereof is administered orally.

5. The method of claim 1 where the amount of seladelpar or a salt thereof is between 1 mg/day and 100 mg/day, when the amount is calculated as seladelpar.

6. The method of claim 5 where the amount of seladelpar or a salt thereof is at least 2 mg/day.

7. The method of claim 5 where the amount of seladelpar or a salt thereof is not more than 50 mg/day.

8. The method of claim 5 where the amount of seladelpar or a salt thereof is 2 mg/day, 5 mg/day, 10 mg/day, 20 mg/day, 50 mg/day, or 100 mg/day.

9. The method of claim 8 where the amount of seladelpar or a salt thereof is 2 mg/day.

10. The method of claim 8 where the amount of seladelpar or a salt thereof is 5 mg/day.

11. The method of claim 8 where the amount of seladelpar or a salt thereof is 10 mg/day.

12. The method of claim 8 where the amount of seladelpar or a salt thereof is 20 mg/day.

13. The method of claim 8 where the amount of seladelpar or a salt thereof is 50 mg/day.

14. The method of claim 8 where the amount of seladelpar or a salt thereof is 100 mg/day.

15. The method of claim 1 where the seladelpar or a salt thereof is administered once/day.

16. The method of claim 1 where the seladelpar or a salt thereof is administered between once/week and every other day.

17. The method of claim 1 that is a method of treating intestinal barrier dysfunction.

18. The method of claim 1 that is a method of treating a disease, other than alcoholic liver disease or NAFLD/NASH, that is associated with intestinal barrier dysfunction.

19. The method of claim 18 where the disease is inflammatory bowel disease, Crohn's disease, irritable bowel syndrome, ulcerative colitis, celiac disease, multiple organ dysfunction syndrome, or chronic viral hepatitis B or C.

20. The method of claim 19 where the disease is irritable bowel syndrome.
